(19) United States
(12) Patent Application Publication
(10) Pub. No.: US 2025/0064150 A1
(43) Pub. Date: Feb. 27, 2025

(54) MASK WHICH USES BODY HEAT TO GENERATE WARMTH

(71) Applicants: Misha MIGDAL, Whitestone, NY (US); Michael BERGESON, San Jose, CA (US)

(72) Inventors: Misha MIGDAL, Whitestone, NY (US); Michael BERGESON, San Jose, CA (US)

(21) Appl. No.: 18/657,267

(22) Filed: May 7, 2024

Related U.S. Application Data
(60) Provisional application No. 63/534,187, filed on Aug. 23, 2023.

(51) Int. Cl. A41D 13/11

(52) U.S. Cl. CPC A41D 13/1138; A41D 13/1176

(57) ABSTRACT

A body heat recovery device includes: a face mask, an inlet tube connected to a first portion of said face mask, an outlet tube connected to a second portion of the face mask; the inlet tube has a filter attached to an end of said inlet tube opposite an end of said inlet tube connected to the face mask to filter debris, dirt, etc., to protect the user.

### CLAIM OF PRIORITY

This application claims priority to Provisional Patent Application Ser. No. 63/534,187 filed on Aug. 23, 2023, the entirety of which is hereby incorporated by reference.

### BACKGROUND OF THE DISCLOSURE

The present disclosure relates to air warming devices. In particular it relates to a mask apparatus worn by a user which uses exhaled air to warm parts of the body.

It has been estimated that about 5.1 million excess deaths per year are associated with non-optimal temperatures. Of those, 4.6 million are associated with colder than optimum temperatures, and 0.5 million are associated with hotter than optimum temperatures.

Heat loss due to respiration is between 25 and 30% of the resting metabolic rate.

Building a Device that recovers heat from respiration and transitions heat back to the torso will save countless lives and makes sleeping out doors tolerable even in temperatures well below zero degrees Celsius.

Previous warming devices are known to generate warm air from body heat to allow the user to breathe warm air, especially in a cold environment. However, many of these devices do not provide the capability of warming parts of the body by using air exhaled by the user.

Accordingly, there is a need for a mask apparatus which uses air exhaled by the user to warm other parts of the body which overcomes deficiencies in existing devices while providing better overall results.

### SUMMARY OF THE DISCLOSURE

The present disclosure relates to air warming devices. In particular it relates to a body heat recovery device.

The half mask is an Elastomeric/Silicone material that makes a comfortable seal between face and mask.

Mask has three one way valves: 2 inlet and 1 outlet.

This allows for inlet air line to be installed on either side of face depending on which side sleeping is preferred.

Fully adjustable Head Gear fits comfortably and securely around front and back of head.

An adjustable elastic band secures lower portion of mask.

Organic Vapor Cartridge Protects You from Organic Debris Including Dirt, Dust, Insects, Paint, Oil, Gasoline Etc.

In accordance with one aspect of the disclosure, the device includes a face mask, an inlet tube with a filter and an outlet tube to bring warm filtered air to any part of the body.

In accordance with another aspect of the disclosure, the face mask has a two way silicone sepsis valve to direct one way airflow on inhalation and exhalation.

In accordance with another aspect of the disclosure, the mask has a silicone pillow that can be inflated or deflated with a syringe.

In accordance with another aspect of the disclosure, a strap is provided for the user's head which has loop and fastener closures for the base of the head and for the crown of the head to ensure a secure fit.

In accordance with another aspect of the disclosure, a fan filter is provided with the inlet tube. Fresh cool air is brought in through the filter protecting the user from air debris as well as mosquitos and other insects.

In accordance with another aspect of the disclosure, a mask has an inlet valve and an outlet valve to bring fresh filtered air from outside of a sleeping bag, a tent, a coat etc. by normal respiratory function and exhaust the warm air to colder parts of the body such as the feet and the torso.

In accordance with another aspect of the disclosure, the device uses warm air exhaled from the lungs to heat other parts of the body.

In accordance with another aspect of the disclosure, the outlet temperature is completely adjustable by changing the speed of the inlet filter fan.

Still other aspects of the disclosure will become apparent upon a reading and understanding of the following detailed description.

### BRIEF DESCRIPTION OF THE DRAWINGS

The disclosure will be better understood in view of the following Figures.

FIG. 1 is a perspective view of the mask assembly in accordance with the preferred embodiment of the present disclosure.

FIG. 2 is a perspective view of the mask and the head gear of the assembly of FIG. 1.

FIG. 3 is a perspective view of the head gear and the inlet and outlet tubes of the assembly of FIG. 1.

FIG. 4 is a perspective view of the mask of the assembly of FIG. 1.

FIG. 5 is a top plan view of the inlet and outlet tubes of the assembly of FIG. 1.

FIG. 6 is a perspective view of the fan filter unit of the assembly of FIG. 1.

FIG. 7 is a perspective view of the head gear of the assembly of FIG. 1.

FIG. 8 is a perspective view of the mask assembly in use with an garment of sleeping bag.

FIG. 9 is a perspective view of the mask in use with a garment.

FIG. 10 is a perspective view of the mask with a closed sleeping bag.

FIG. 11 is a front elevated view showing the mask being used with a user without a filter.

FIG. 12 is a front elevated view showing the mask being used with a user with a filter.

FIG. 13 is a perspective view of a temperature gage illustrating outdoor temperature and temperature with the mask.

FIG. 14 is another perspective view of a temperature gage illustrating outdoor temperature and temperature within the mask.

FIG. 15 is another perspective view of a temperature gage illustrating outdoor temperature and temperature within the mask.

### DETAILED DESCRIPTION OF THE PREFERRED EMBODIMENTS

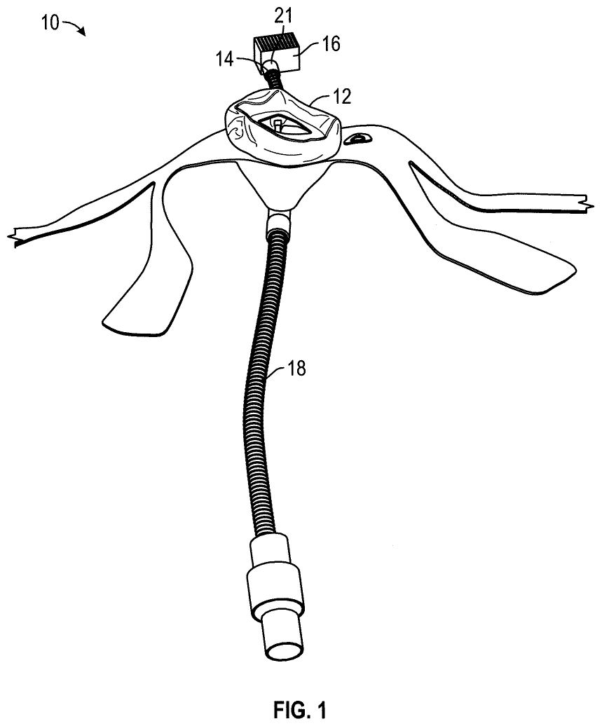

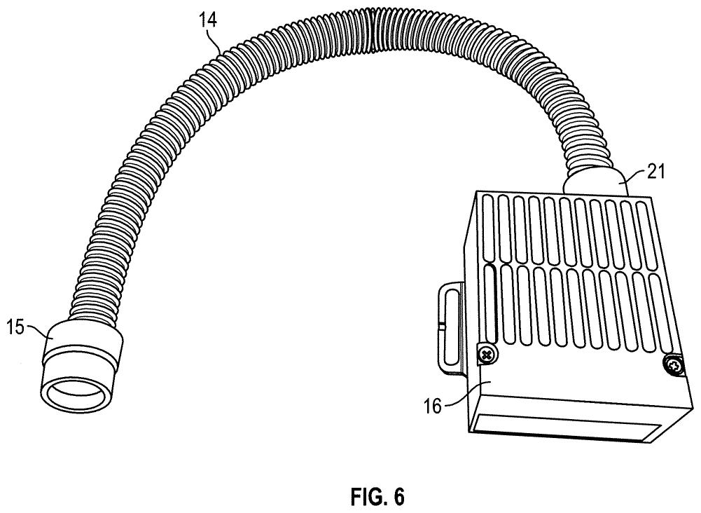

With reference now to the drawings, in particular FIGS. 1-15, a mask apparatus 10 for using exhaled warm air to warm other parts of the body will now be described in accordance with a preferred embodiment of the disclosure. Referring to FIG. 1, the mask apparatus or body heat recovery device 10 includes a face mask 12, an inlet tube 14 with a fan and filter assembly 16, attached on a distal and of a tube and an outlet tube 18. The outlet tube can have varying lengths in order to bring warm filtered air to any part of the body.

The temperature data using body heat recovery device: Outside Temp 16 F/−9 C 25.7 F/3.5 C 37 F/3 C Inside sleeping bag: 64 F/18 C 68 F/20 C 74 F/23 C

The mask itself is preferably made of an elastomeric/Silicone material that makes a comfortable seal between the nose, face and the mouth.

The mask has three one way valves: two inlet valves and one outlet valve.

This allows for the inlet air line to be installed on either side of the face depends on which sleeping side is preferred.

Fully adjustable head gear fits comfortably and securely around the front and the back of the head.

An adjustable elastic band secures the lower portion of the mask.

An organic vegan cartridge protects the user from organic debris, such as dust, dirt, insects, paint, oil, gasoline, etc.

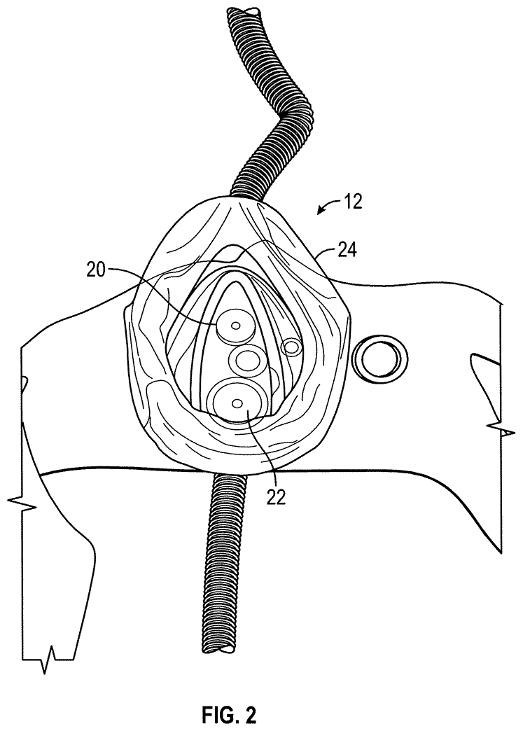

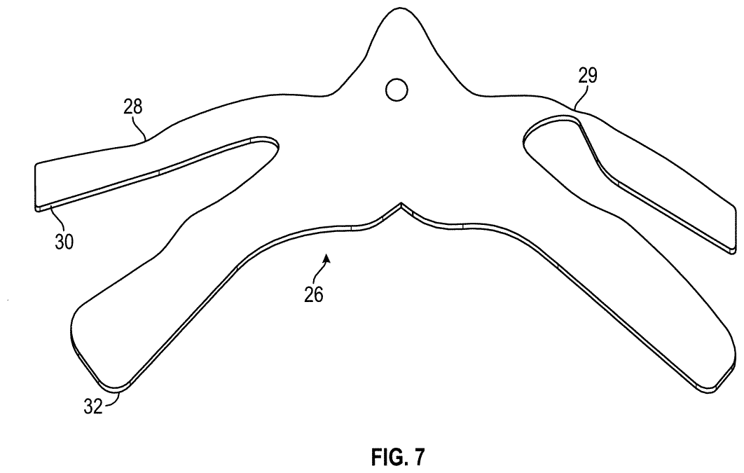

Referring now to FIGS. 2 and 4, the face mask 12 has two one-way silicone sepsis valves 20, 22 to direct one-way airflow on inhalation and exhalation. Alternatively, a second one-way elastic valves 23 can be provided on the opposite side of the mask. The mask also preferably includes a silicone pillow 24 that can be inflated or deflated with a syringe (not shown). This allows for an air-tight seal to be formed around the user's nose, cheeks and mouth. Referring to FIG. 7, head gear 26 has straps 28,29 with two loops and fastener (i.e. Velcro) closures 30, 32: one for the base of the head and one for the crown of the head to ensure a secure fit.

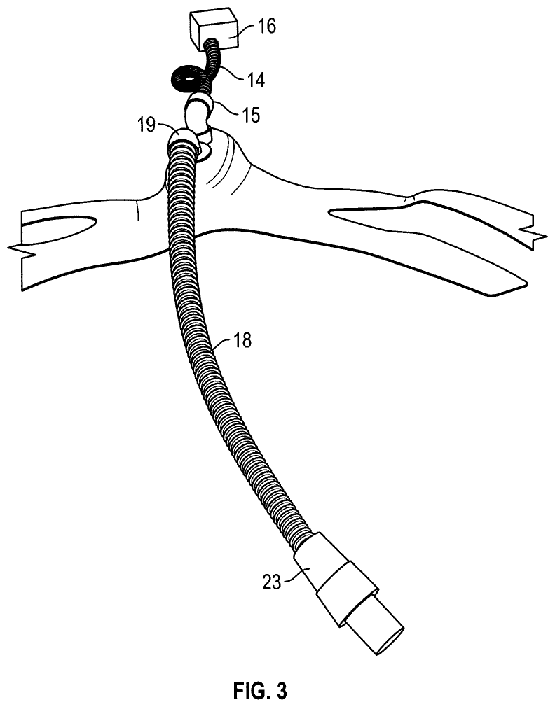

Referring to FIGS. 3,5 and 6, the inlet tube 14 and fan filter 16 preferably includes an 18 inch long inlet tube having quick release connectors 15, 21 and a 360 degree rotation, which allows the hose to “spin” and avoid tangles and ensures smooth airflow. The inlet tube 14 and fan filter 16 swivels and seals correctly, effectively preventing leakage and hissing noise.

The filter 16 preferably includes a HEPA H13 high-efficiency filter, and a three speed adjustable fan which provides a continuous stream of fresh air to be sent to the mask. Fresh cool air is drawn in through the filter protecting the user from air debris, or mosquitos or other insects. Inlet air flows through the inlet tube 14, through a one way valve and into the user's lungs.

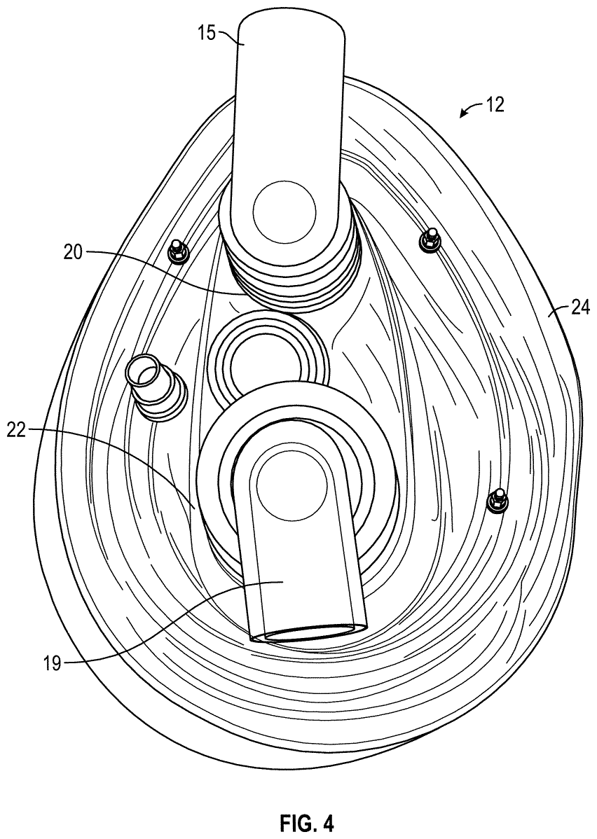

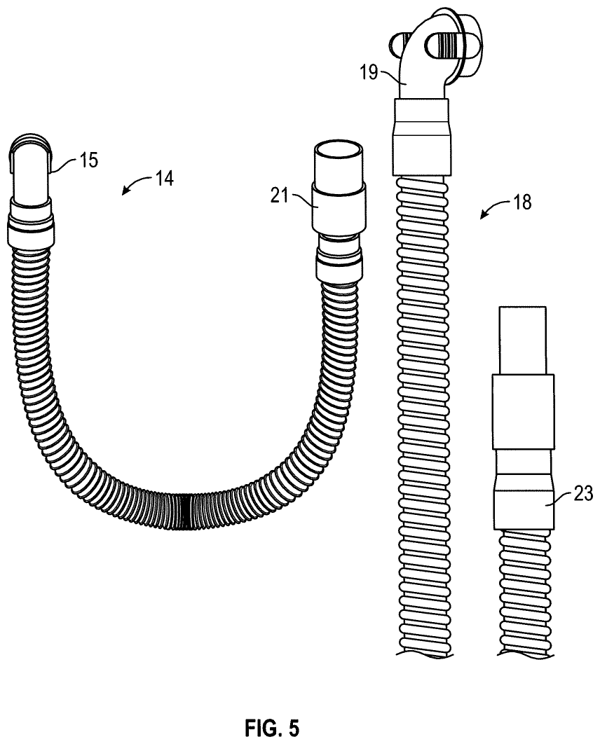

Fresh air is then heated by normal respiratory function and is exhausted through a one way outlet valve. Outlet tube 18 then carries the heated air to other parts of the body specified by the user. Referring to FIGS. 3 and 5, the outlet tube 18 preferably can be 18 inches or 36 inches long, and also comes with a quick release connector 19 and connector 23. This tube also has 360 degree rotation, so it can “spin” to help pivot the hose to avoid tangles and ensure smooth airflow, and swivels and seals correctly, effectively preventing leakage and hissing noise. The outlet temperature can be adjusted by simply changing the speed of the inlet filter fan.

The mask apparatus or body heat recovery device uses the warm air that is exhaled from the user's lungs to heat other parts of the body, such as arms, torso, etc. Approximately 30 percent of human body heat is lost in normal respiratory function. The apparatus enables the user to recover and re-use some of the lost body heat.

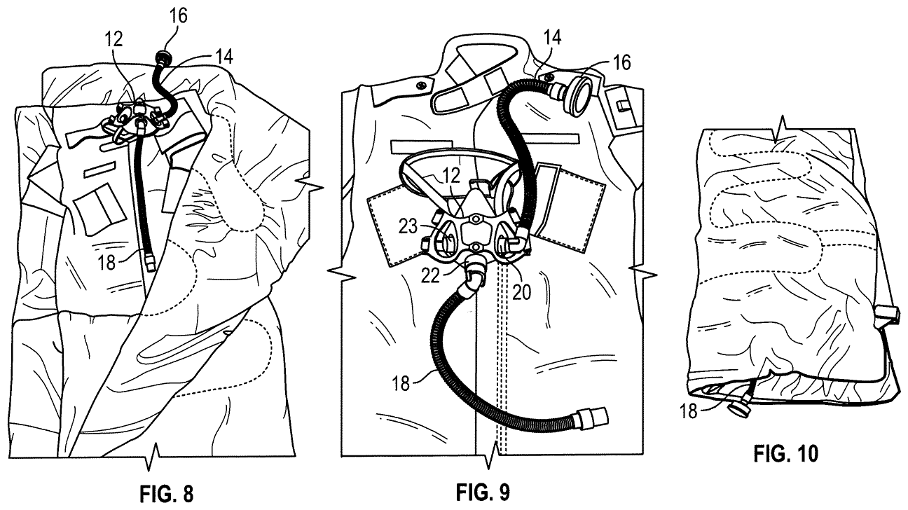

Referring to FIGS. 8-10 several applications of the mask are shown refer to FIG. 8, the mask is shown being used with a garment 100 and a sleeping bag 102—

In FIG. 9 the mask is shown being used with a garment 100.

In FIG. 10 the mask is shown being used inside a sleeping bag 102.

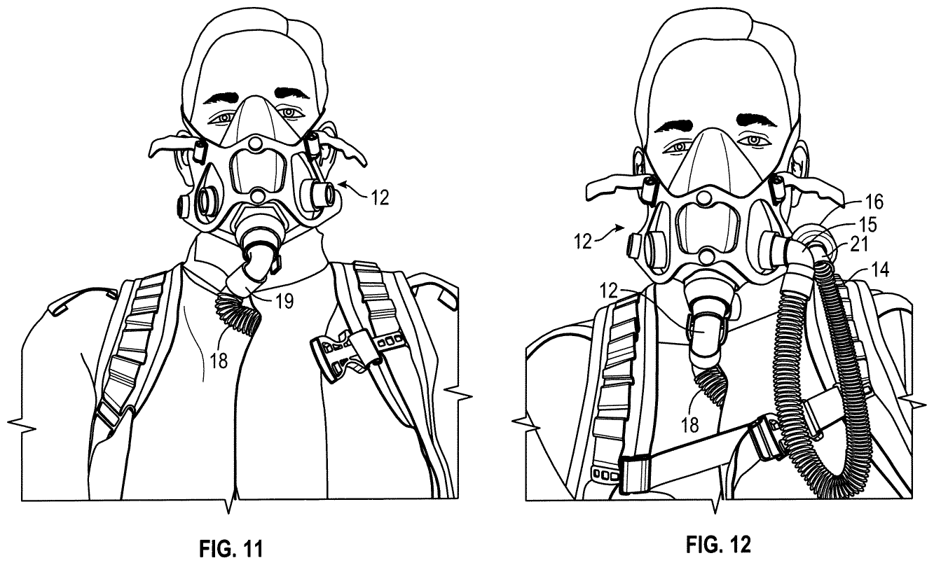

FIG. 11 illustrates the mask being used with a filter.

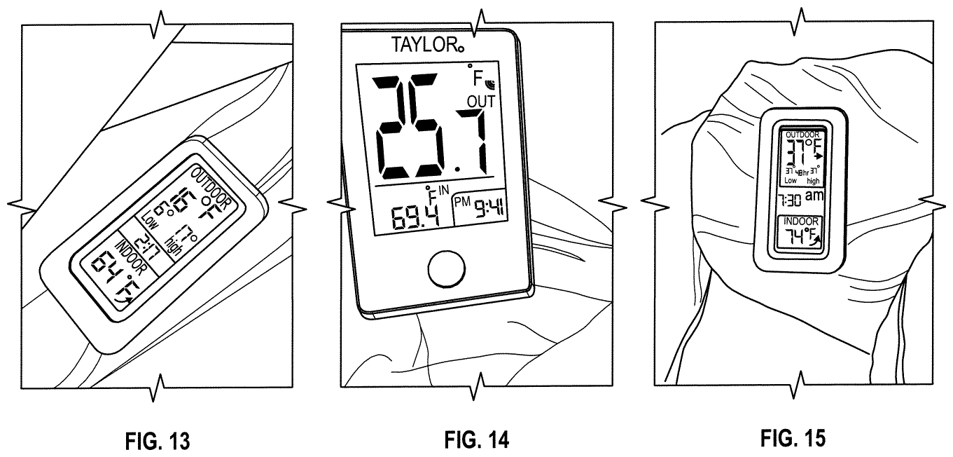

FIG. 12 illustrates the mask being used with the filter of outlet tube. Some data that has resulted from the body heat recovery device being used with a sleeping bag is shown in FIGS. 13,14,15.

FIG. 13 shows a temperature gage where the outside temperature is 16° F. (and −9° C.) Where the inside temperature (with the sleeping bag) is 64° F. (18° C.)

FIG. 14 shows a temperature gage where the outside temperature is 25.7° F. (−3.5° C.) Where the inside temperature (inside the sleeping bag) is 68° F. (20° C.)

FIG. 15 shows a temperature gage where the outside temperature is 37° F. (3° C.) where the inside temperature (i.e.) inside the sleeping bag is 74° F. (23° C.)

Using the mask with one inlet valve and one outlet valve allows the user to bring fresh filtered air from outside of the sleeping bag, a tent or other enclosure, a coat or other garment and heat the air by normal respiratory function and exhaust the warm air to other parts of the body such as the arms, feet or torso area.

The exemplary embodiments have been described with reference to the preferred embodiment. Obviously, modifications and alterations will occur to others upon a reading and understanding of the preceding detailed description. It is intended that the exemplary embodiments be construed as including all such modifications and alterations insofar as they come within the scope of the above detailed description and appended claims.

1. A body heat recovery device comprising a face mask and inlet tube connected to a first portion of said face mask an outlet tube connected to second portion of said face mask, wherein said inlet tube has a filter attached to an end of said inlet tube opposite on end of said inlet tube connected to said face mask.

2. The body heat recovery device of claim 1, wherein said face mask comprises further comprising a front one-way valve from and inlet tube and a one way second for said outlet tube the direct one-way airflow from inhalation to exhalation.

3. The body heat recovery device of claim 1, wherein said face mask comprises an inflatable pillow to form a seal around a user's face.

4. The body heat recovery device of claim 1, wherein said face mask further comprising straps for securing the face mask to user's face.

5. The body heat recovery device of claim 1, wherein said inlet tube has a guard release connection to the said filter.

6. The body heat recovery device of claim 1, wherein the further comprising NEPA N13 high efficiency filter

7. The body recovery device of claim 1, wherein said inlet tube and filter further comprising a fan.

8. The body recovery device of claim 7 wherein said face is a three speed adjustable fan.

9. The body recovery device of claim 1, wherein said outlet tube has a quick release connector attached said outlet tube the said face mask.

10. The body recovery device of claim 1, wherein said face mask comprising an elastomeric and silicone material.

11. The body recovery device of claim 2, further comprising a third one-way valve on an opposite side of the face mask from the front one way valve.
